(19) United States
(12) Patent Application Publication
(10) Pub. No.: US 2008/0255433 A1
(43) Pub. Date: Oct. 16, 2008

(54) OPTOACOUSTIC MONITORING OF MULTIPLE PARAMETERS

(75) Inventors: Donald S. Prough, Galveston, TX (US); Rinat O. Esenaliev, League City, TX (US); Donald Deyo, Galveston, TX (US); Yuriy Y. Petrov, Galveston, TX (US); Irina Y. Petrova, Galveston, TX (US)

Correspondence Address: ROBERT W STROZIER, P.L.L.C, PO BOX 429, BELLAIRE, TX, 77402-0429, US

(73) Assignee: The Board of Regents of The University of Texas Syatem, Austin, TX (US)

(21) Appl. No.: 12/101,891

(22) Filed: Apr. 11, 2008

Related U.S. Application Data
(60) Provisional application No. 60/911,193, filed on Apr. 11, 2007.

(51) Int. Cl. A61B 5/145

(52) U.S. Cl. 600/301; 600/310

(57) ABSTRACT

An optoacoustic technique for absolute, accurate, continuous, and real-time measurement of a variety of important diagnostic variables is disclosed, where the variables include: (1) noninvasive measurements of circulating blood volume (BV) and cardiac output (CO); (2) calculation from the measured variables of cardiac index (CI) and systemic oxygen delivery (DO₂); (3) concentrations of hemoglobin derivatives (e.g., carboxyhemoglobin [HbCO], reduced hemoglobin [Hb], oxygenated hemoglobin [HbOxy], and methemoglobin [HbMet]), total hemoglobin concentration [THb], concentrations of lactate, myoglobin, cholesterol, body pigments, and exogenous dyes; (4) content in tissues of water, fat, protein, calcium, and blood; as well as density of hard and soft tissues; and (5) accurate noninvasive measurement of blood pressure (or vascular pressure) in arteries, arterioles, veins, capillaries, using occlusion-induced changes in optoacoustic signal induced in blood circulating in the vessels. The optoacoustic technique can be used for single measurement, continuous measurement, or continuous monitoring of these variables.

### RELATED APPLICATIONS

This application claims priority to and benefit of U.S. Provisional Patent Application Ser. No. 60/911193 filed Apr. 11, 2007.

### BACKGROUND OF THE INVENTION

1. Field of the Invention

The present invention relates to use the optoacoustic technique for absolute, accurate, continuous, and real-time measurement of a variety of important diagnostic variables.

More particularly, the present invention relates to use the optoacoustic technique for absolute, accurate, continuous, and real-time measurement of a variety of important diagnostic variables including: (1) noninvasive measurements of circulating blood volume (BV) and cardiac output (CO); (2) calculation from the measured variables of cardiac index (CI) and systemic oxygen delivery (DO₂); and (3) concentrations of hemoglobin derivatives (e.g., carboxyhemoglobin [HbCO], reduced hemoglobin [Hb], oxygenated hemoglobin [HbOxy], and methemoglobin [HbMet]), total hemoglobin concentration [THb], concentrations of lactate, myoglobin, cholesterol, body pigments, and exogenous dyes; (4) content in tissues of water, fat, protein, calcium, and blood; as well as density of hard and soft tissues; and (5) accurate noninvasive measurement of blood pressure (or vascular pressure) in arteries, arterioles, veins, capillaries, using occlusion-induced changes in optoacoustic signal induced in blood circulating in the vessels. The optoacoustic technique can be used for single measurement, continuous measurement, or continuous monitoring of these variables.

2. Description of the Related Art

Recently, we proposed to use optoacoustic technique for monitoring of total hemoglobin concentration [THb] (see U.S. Pat. No. 6,751,490, incorporated herein by reference) and blood oxygenation [HbOxy] (see U.S. Pat. No. 6,498,942, incorporated herein by reference).

Even with these advances in non-invasive optoacoustic techniques, there is still a need in the art for an optoacoustic technique for absolute, accurate, continuous, and real-time measurement of a variety of important diagnostic variables. Moreover, in this application we propose to measure peak-to-peak amplitude of optoacoustic signals and first derivative of normalized optoacoustic signals for more accurate, sensitive, and specific monitoring the diagnostic variables including [THb] and [HbOxy].

### SUMMARY OF THE INVENTION

The present invention provides for the use of an optoacoustic technique for absolute, accurate, continuous, and real-time measurement of a variety of important diagnostic variables including: (1) noninvasive measurements of circulating blood volume (BV) and cardiac output (CO); (2) calculation from the measured variables of cardiac index (CI) and systemic oxygen delivery (DO₂); and (3) concentrations of hemoglobin derivatives (e.g., carboxyhemoglobin [HbCO], reduced hemoglobin [Hb], oxygenated hemoglobin [HbOxy], and methemoglobin [HbMet]), total hemoglobin concentration [THb], concentrations of lactate, myoglobin, cholesterol, body pigments, and exogenous dyes; (4) content in tissues of water, fat, protein, calcium, and blood; as well as density of hard and soft tissues; and (5) accurate noninvasive measurement of blood pressure (or vascular pressure) in arteries, arterioles, veins, capillaries, using occlusion-induced changes in optoacoustic signal induced in blood circulating in the vessels. The optoacoustic technique can be used for single measurement, continuous measurement, or continuous monitoring of these variables.

### BRIEF DESCRIPTION OF THE DRAWINGS

The invention can be better understood with reference to the following detailed description together with the appended illustrative drawings in which like elements are numbered the same:

FIG. 1A depicts a schematic diagram of an embodiment of an optoacoustic system of this invention.

FIG. 1B depicts a schematic diagram of an embodiment of an optoacoustic probe of this invention.

FIGS. 2A-D depict optoacoustic signals from the skin (the first peak) and the radial artery (the second peak) of a healthy volunteer at wavelengths of 700, 850, 975 and 1064 nm. The depth (upper horizontal axis) is in reference to the skin surface and is linearly proportional to time.

FIG. 3A depicts a peak-to-peak amplitudes of the optoacoustic signals from the skin (open triangles) and from the radial artery (solid circles) at different wavelengths.

FIG. 3B depicts a corrected arterial peak-to-peak amplitude at different wavelengths.

FIG. 4 depicts optoacoustic signals measured with the laser-diode-based optoacoustic system from sheep blood with varying total hemoglobin concentration.

FIG. 5 depicts a peak-to-peak amplitude of the optoacoustic signal measured with the laser-diode-based optoacoustic system from the radial artery phantom vs. directly measured total hemoglobin concentration in blood.

FIG. 6 depicts an optoacoustic signal from the radial artery of a healthy volunteer obtained with the laser-diode-based system (k=905 nm).

FIG. 7A depicts a typical optoacoustic signals measured from the sheep SSS in vivo at 1064 nm.

FIG. 7B depicts a typical optoacoustic signals measured from the sheep SSS in vivo at 700 mm.

FIG. 8A depicts a SSS blood oxygenation (black) and optoacoustic signal amplitude (grey) at 1064 nm during 2 cycles of changes in blood oxygenation.

FIG. 8B depicts a SSS blood oxygenation (black) and optoacoustic signal amplitude (grey) at 700 nm during 2 cycles of changes in blood oxygenation.

FIG. 9A depicts an optoacoustic signal amplitudes measured from the sheep SSS in vivo at 1064 nm and 700 nm during the first cycle.

FIG. 9B depicts an optoacoustic signal amplitudes measured from the sheep SSS in vivo at 1064 nm and 700 nm during the second cycle.

FIG. 10A depicts a correlation between optoacoustically predicted and actual SSS blood oxygenation during the first cycle.

FIG. 10B depicts a correlation between optoacoustically predicted and actual SSS blood oxygenation during the second cycle.

FIG. 11A depicts a standard deviation and bias between optoacoustically predicted and actual SSS blood oxygenation during the first cycle.

FIG. 11B depicts a standard deviation and bias between optoacoustically predicted and actual SSS blood oxygenation during the second cycle.

FIG. 12A depicts a SSS blood oxygenation (black) and optoacoustic signal amplitude (grey) at 1064 nm measured with minimal motion artifacts.

FIG. 12B depicts a SSS blood oxygenation (black) and optoacoustic signal amplitude (grey) at 700 nm measured with minimal motion artifacts.

FIG. 13A depicts a correlation between optoacoustically predicted and actual SSS blood oxygenation measured with minimized motion artifacts.

FIG. 13B depicts a standard deviation and bias between optoacoustically predicted and actual SSS blood oxygenation measured with minimized motion artifacts.

FIG. 14A depicts an optoacoustic signals from arterial blood with different [THb]s.

FIG. 14B depicts first derivatives of the normalized optoacoustic signals from blood with different [THb]s.

FIG. 14C depicts a maximum derivative of the optoacoustic signals from blood in vitro (open triangles) and from radial arteries of two volunteers (solid triangles).

FIG. 15A depicts an optoacoustic signal amplitude measured from the radial artery phantom with blood at [THb]=15.0 g/dL vs. lateral displacement of the probe.

FIG. 15B depicts an optoacoustic system with a probe scanning over the radial artery that can be used for monitoring of the physiologic variables.

FIG. 15C depicts an array-based optoacoustic system for monitoring of physiologic variables in the arterial blood (in the radial artery) and in the venous blood (in the internal jugular vein). An optoacoustic system without array or scanning and only with one acoustic detector for monitoring of physiologic variables such as content of water, myoglobin, lactate, fat, protein, calcium, etc., is shown as well.

FIG. 16A depicts optoacoustic signals recorded from blood at different ICG concentration.

FIG. 16B depicts an amplitude of optoacoustic signals recorded from blood at different ICG concentration.

FIG. 16C depicts a peak-to-peak amplitude of optoacoustic signals recorded from blood at different ICG concentration.

FIG. 16D depicts an effective attenuation coefficient of blood measured using the optoacoustic signals recorded blood at different ICG concentration.

FIG. 17A depicts peak-to-peak amplitude measured in real time from the radial artery during variation of external pressure exerted by the optoacoustic probe.

FIG. 17B depicts peak-to-peak amplitude measured in real time from a wrist vein during variation of external pressure exerted by the optoacoustic probe.

### DETAILED DESCRIPTION OF THE INVENTION

The inventors have found that optoacoustic techniques can be used for absolute, accurate, continuous, and real-time measurement of a variety of important diagnostic variables including: (1) noninvasive measurements of circulating blood volume (BV) and cardiac output (CO); (2) calculation from the measured variables of cardiac index (CI) and systemic oxygen delivery (DO₂); and (3) concentrations of hemoglobin derivatives (e.g., carboxyhemoglobin [HbCO], reduced hemoglobin [Hb], oxygenated hemoglobin [HbOxy], oxygenated hemoglobin [HbOxy], and methemoglobin [HbMet]), total hemoglobin concentration [THb], concentrations of lactate, myoglobin, cholesterol, body pigments, and exogenous dyes; (4) content in tissues of water, fat, protein, calcium, and blood; as well as density of hard and soft tissues; and (5) accurate noninvasive measurement of blood pressure (or vascular pressure) in arteries, arterioles, veins, capillaries, using occlusion-induced changes in optoacoustic signal induced in blood circulating in the vessels. Current techniques are invasive, require blood sampling and analysis, and cannot be performed in real time or near real time and continuously over a long period of time or over a sustained period of time. The optoacoustic technique can be used for single measurement, continuous measurement, or continuous monitoring of these variables. The term “real time” means that results are displayed after acquisition with a delay of not more the 5 s (in other embodiments: less than 1 s, less than 0.5 s, near instantaneous); while the term “near real time” means that the results are displayed after acquisition with a delay of less than 1 minute (in other embodiments: less than 30 s, less than 20 s, less than 10 s and less than 5 s). The system can be designed to measure or monitor at least one or all of the parameters on a single measurement, an intermediate measurement, a periodic measurement, a semi-continuous, or a continuous measurement basis.

The wavelength range for optoacoustic monitoring of these variables is between 200 nanometers and 20,000 nanometers, and in other embodiments, between 600 and 2,000 nanometers. The pulse duration for optoacoustic monitoring of these variables is between 1 femtosecond and 10 microseconds, and in other embodiments, between 0.1 and 200 nanoseconds. The optoacoustic probe can be places on skin surface or introduced in hollow organs. For instance, for monitoring of blood parameters in pulmonary artery or aorta one can insert an optoacoustic probe in the esophagus.

The optoacoustic technique is based on generation of ultrasound (optoacoustic) waves by pulsed light and detection of these waves by sensitive acoustic transducers. The optoacoustic technique has high (optical) contrast and high (ultrasound) resolution and can be used for direct probing of blood vessels and monitoring of blood parameters. Hemoglobin is a major blood chromophore in the visible and near IR spectral range that allows for accurate measurement of concentrations of [THb], [HbOxy], as well as [Hb], [HbCO], [HbMet], and other hemoglobin derivatives. Tissues contain other chromophores such as water, myoglobin, lactate, fat, protein, calcium. Therefore, optoacoustic technique can be used for monitoring of these physiologic variable as well. For instance, water has absorption peaks at 970, 1200, 1450 nm and strong absorption at longer wavelengths. Therefore, one can use optical pulses at these and near these to measure and monitor water content in tissue. Fat has an absorption peak at 1210 nm (range: 1120 to 1250 nm), while protein has absorption in the range of 1100 and 1230 nm. Therefore, one can use these wavelengths to measure optoacoustically content of fat or protein in tissues.

Moreover, injection of indocyanine green (ICG) dye and direct measurement of ICG kinetics in arteries may provide highly accurate monitoring of CO and measurement in veins may provide highly accurate monitoring of CO, CI, and (BV). CI, combined with the [THb] and [HbOxy] measurements, yields DO₂. Measurement of venous [HbOxy] permits assessment of the adequacy of DO₂ to meet oxygen demand. Accurate and continuous measurement of these parameters will improve clinical outcome and reduce morbidity in a variety of conditions in outpatients, including those with chronic heart failure, in inpatients, such as critically ill and surgical patients, and in the field, emergency, and mass casualty settings.

### Importance of Real-Time or Near Real-Time Continuous Monitoring

Large populations of patients need monitoring of important diagnostic variables including: circulating blood volume (BV), cardiac output (CO) and index (CI), oxygen delivery (DO₂), venous oxyhemoglobin saturation ([HbOxy]), concentration of hemoglobin derivatives including carboxyhemoglobin and methemoglobin and noninvasive measurement of blood pressure. At present, no accurate, robust, noninvasive, real-time or near real-time continuous monitoring of these parameters is available.

The optoacoustic technique is also ideal for noninvasive quantification of the blood concentrations of other chromophores such as indocyanine green (ICG). Noninvasive quantification of ICG concentrations ([ICG]) provides a clinically relevant way to quantify CO, CI (CO divided by body surface area), BV and hepatic clearance of ICG as an index of hepatic perfusion. Measurement of CI, [THb] and [HbOxy] permits calculation of DO₂, which is a powerful prognostic indicator in critically ill patients and which has been successfully used as an endpoint to improve clinical outcome in critically ill patients, including patients admitted to emergency departments with sepsis.

Measurement of CO has become a standard technique in the care of critically ill patients and patients undergoing major surgical procedures, such as cardiac surgery and vascular surgery. Clinically useful CO measurements first became possible in the mid-1970's with the introduction of thermodilution, pulmonary arterial catheters[1]. At present, the annual expenditures on pulmonary arterial catheters in the United States are estimated at $2,000,000,000 [2]. However, vigorous debate has continued about the influence of pulmonary arterial catheterization on clinical outcome, at least in part because the risk of performing an invasive procedure partially or completely offsets the clinical value of monitor-guided therapy [3]. Because the clinical framework for hemodynamic monitoring is now widespread, clinical introduction of a noninvasive monitor of CO could proceed rapidly.

Noninvasive monitoring of CO permits calculation of certain highly important variables. Adjustment of CO measurements for body size requires calculation of CI, which is derived by dividing CO by body surface area. Multiplication of CI (or CO) by [THb] and percent [HbOxy] permits calculation of DO₂, which correlates powerfully with clinical outcome in critically ill patients [4] and which has reduced mortality and morbidity when used as an effective endpoint for resuscitation of high-risk surgical patients [5]. Noninvasive measurement and calculation of DO₂ will facilitate goal-directed hemodynamic therapy without the risk associated with invasive pulmonary arterial catheterization.

Injection of ICG for measurement of CO permits subsequent calculation of BV and hepatic clearance of ICG (a surrogate for hepatic perfusion and function). The CO is calculated from the early (<60 seconds) peak and decline in arterial ICG concentration after venous injection. Extrapolation to the initial dilution volume of ICG (<120 sec) permits calculation of BV, while subsequent clearance (30-60 minutes) reflects hepatic perfusion and function. Rapidly changing arterial concentrations, obtained by optoacoustic measurements in the radial artery, are best for determining CO, while BV and hepatic clearance can be determined from optoacoustic measurements over large veins.

Current clinical measurement of blood pressure is accomplished using a noninvasive manual method (blood pressure cuff inflation and deflation while listening with a stethoscope for Korotkoff sounds), a noninvasive automated method or direct measurement using an arterial catheter. In patients in shock, the noninvasive manual method is tedious and distracts health care providers from other essential tasks. However, the noninvasive automated methods are inaccurate in low blood pressure states, often substantially overestimating blood pressure. This deficiency has prompted recommendations that the manual method be used in patients in shock. Direct measurements are not practical until patients are admitted to emergency departments, intensive care units or operating rooms and until they are stabilized. In such situations, an accurate, automated noninvasive method would greatly facilitate patient care. Noninvasive optoaocoustic measurement of blood pressure will address this clinical need. Optoacoustic measurement of blood pressure is dependent on changes in arterial diameter that occur with occlusion by a blood pressure cuff. With occlusion of an artery, the [THb] signal is markedly reduced; as pressure in the cuff is reduced below systolic blood pressure, the [THb] signal abruptly increases in magnitude and as the pressure in the cuff is reduced toward diastolic blood pressure the shape of the time-resolved optoacoustic signal changes characteristically, i.e., the oscillation of the signal markedly decreases. Unlike other noninvasive blood pressure measurements, optoacoustic measurement of blood pressure is minimally influenced by low blood pressure, as in hemorrhagic shock, or high-noise environments, such as emergency transport vehicles.

### Explanation of Optoacoustic Techniques

Absorption of light energy in a medium is followed by rapid thermal relaxation and a temperature increase in the medium. Thermal expansion of the irradiated medium induces mechanical stress (pressure rise). This mechanism is referred to as the thermo-optical mechanism of pressure generation. A short optical pulse with the incident fluence, Fₒ, induces a pressure rise, P(z), in the medium upon condition of stress confinement:

P( [symbol] )=(βcₛ²/Cₚ)μₐF=ΓμₐF( [symbol] )=ΓμₐFₒ exp(−μₐ [symbol] ) (1)

where b [1/° C.] is the thermal expansion coefficient; cₛ [cm/s] is the speed of sound; Cₚ [J/g° C.] is the heat capacity at constant pressure; F(z) [J/cm²] is the fluence of the optical pulse; and μₐ [cm⁻¹] is the absorption coefficient of the medium. The optoacoustic pressure in Eq. 1 can be expressed in J/cm³ or in bar (1 J/cm³=10 bar). The expression (bcₛ²/Cₚ) in Eq. 1 represents the dimensionless Grüneisen parameter, G. The exponential attenuation of the optical radiation in the medium is represented by exp(−μₐz).

The condition of stress confinement means that there is insignificant stress relaxation in the irradiated volume during the optical pulse. To provide this condition, the duration of the optical pulse should be shorter than the time of stress propagation out of the irradiated volume. Nanosecond laser pulses can be used to generate conditions of stress confinement for many optoacoustic applications including monitoring of the blood parameters.

According to Eq. 1, optoacoustic pressure amplitude is proportional to the Grüneisen parameter, fluence, and absorption coefficient of the medium, while the pressure spatial profile is dependent on the absorption coefficient.

Since z and t are related by the simple equation:

z=cₛt (2)

the spatial distribution of laser-induced pressure P(z) is detected by a wide-band transducer as a temporal profile P(t):

P(t)=ΓμₐFₒ exp(−μₐcₛt) (3)

Therefore, by recording and analyzing the amplitude or temporal profile of optoacoustic waves, one can measure the absolute value of the absorption coefficient of the irradiated medium.

Most tissues are strongly scattering media in the visible and near-IR spectral range. Three major optical parameters are responsible for distribution of light in tissues: the absorption (μₐ), scattering (μₛ), and effective attenuation (μ_{eff}) coefficients. The effective attenuation coefficient can be expresses as:

μ_{eff}={3μₐ[μₐ+μₛ(1−g)]}^{1/2} (4)

where μₛ(1−g) is the reduced scattering coefficient, μₛ. Light penetration depth in tissues is defined as 1/μ_{eff}. Absorption and reduced scattering coefficients of tissues are low in the near-IR spectral range (from 600 to 1300 nm), which results in deeper penetration of near-IR radiation compared with that of other parts of the spectrum. Application of near-IR radiation will allow sufficient penetration of light in tissues for optoacoustic monitoring of the blood parameters.

Hemoglobin has a high absorption coefficient in the visible and near-IR spectral range. Therefore, both the amplitude and spatial distribution of the generated optoacoustic pressure induced in blood are dependent on concentrations chromophores such as ICG and the hemoglobin derivatives. High z-axial resolution of the optoacoustic technique permits direct measurement of these parameters because the optoacoustic waves induced in blood arrive at the acoustic transducer at the time defined by Eq. 2.

Therefore, by recording and analyzing the amplitude and temporal profile of optoacoustic pressure signals, one can monitor these parameters with high accuracy. The high z-axial resolution of the optoacoustic technique will permit direct measurement of these parameters, because the signal from the blood will arrive at the acoustic transducer at the time defined by Eq. 2. Due to optoacoustic wave diffraction during propagation to the detecting transducer or optoacoustic probe, the optoacoustic wave becomes bipolar and signal detected by the probe is typically bipolar and has a positive peak (a maximum) and a negative peak (a minimum). We propose to measure peak-to-peak amplitude of the biopolar signals that is the magnitude of the signal measured between the maximum and the minimum. Often, measurement of the amplitude (the magnitude of the positive peak) is difficult due to a signal offset and overlying tissue signal, while the measurement of peak-to-peak amplitude is easier and provides accurate monitoring of these variables.

Moreover, variation of overlying tissue properties (both optical, acoustical, and geomertical) may reduce accuracy, sensitivity, and specificity of optoacoustic monitoring. To minimize the influence of these effects on accuracy, sensitivity, and specificity of optoacoustic monitoring, one can use measurement of first derivative of normalized optoacoustic signal. In contrast to amplitude, the first derivative of the normalized optoacoustic signal has no or minimal dependence on the overlying tissue properties.

### EXPERIMENTS OF THE INVENTION

Current clinical measurement of blood analytes and exogenous substances circulating in blood requires withdrawal of a blood sample that is then analyzed using a point-of-care device or must be transported to a clinical laboratory. Blood sampling is invasive, cannot be continuous, and requires expensive personnel time and attention. In addition, delay between blood sampling and reporting of results interferes with timely decision making. Repeated blood sampling during surgery or in critically ill patients contributes to loss of red blood cells and aggravates anemia.

We disclosed in U.S. Pat. Nos. 6,751,490 and 6,498,942 and demonstrated the feasibility of noninvasive measurement of [THb] and oxygenation using an optoacoustic technique^{3,4} Here, we disclose means for:
1) optoacoustic monitoring of important diagnostic parameters;
   2) optimization of optoacoustic monitoring of these important parameters as well as [THb] and oxygenation using:
   a) measurement of peak-to-peak amplitude instead of the amplitude;
   b) measurement of first derivative of normalized signals;
   c) scanning of optoacoustic probe to minimize influence of motion artifacts;
   d) identifying most preferable spectral range for monitoring of these parameters;
   e) laser diode-based systems which have low cost, weight, and compact dimensions;
   f) novel piezo-materials for optoacoustic probes such as ceramics with very low planar coupling coefficient which provide highly sensitive, wide-band detection with minimal acoustic ringing.

One convenient blood vessel for optoacoustic monitoring of a variety of these parameters is the radial artery, which is located within a few millimeters of the skin surface on the ventrolateral wrist. We identified a spectral range most suitable for detection of optoacoustic signals from radial artery and other blood vessels. For optoacoustic monitoring of the blood parameters, a strong and clear arterial signal is highly desirable. At the same time, the signal generated in the overlying tissues reaches the transducer first and may distort the later-arriving arterial signal by causing multiple reverberations within the transducer. Besides, absorption of light by skin reduces laser fluence that reaches radial artery. Thus, in our search for an optimum wavelength range for blood vessel probing, we were looking for wavelengths where the skin signal had minimal amplitude. Higher amplitude of the arterial signal served as an additional criterion.

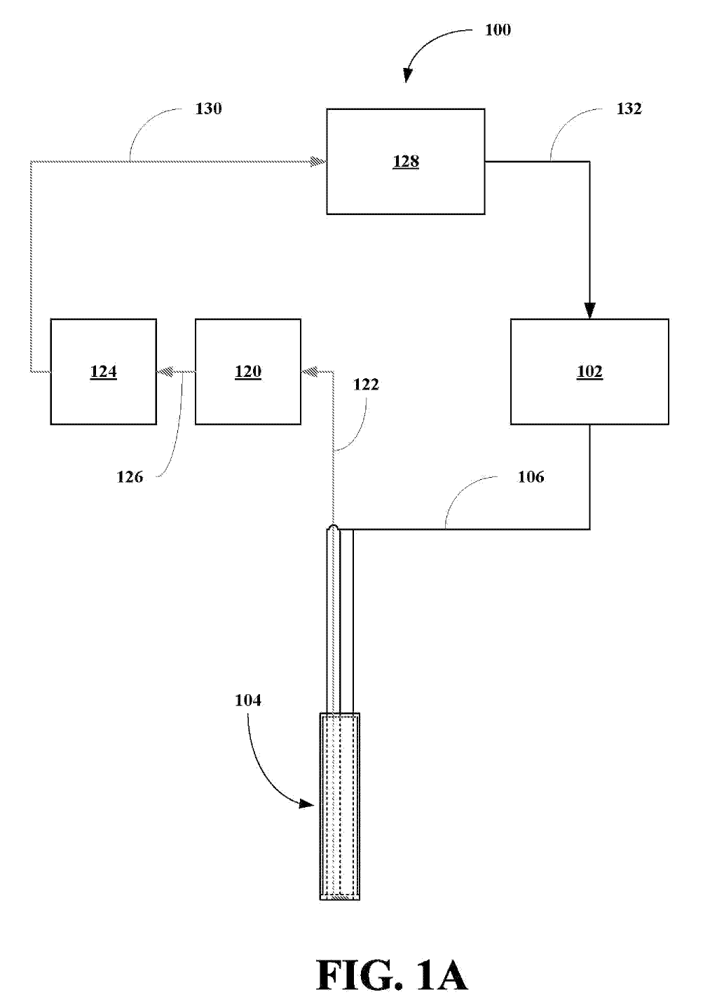

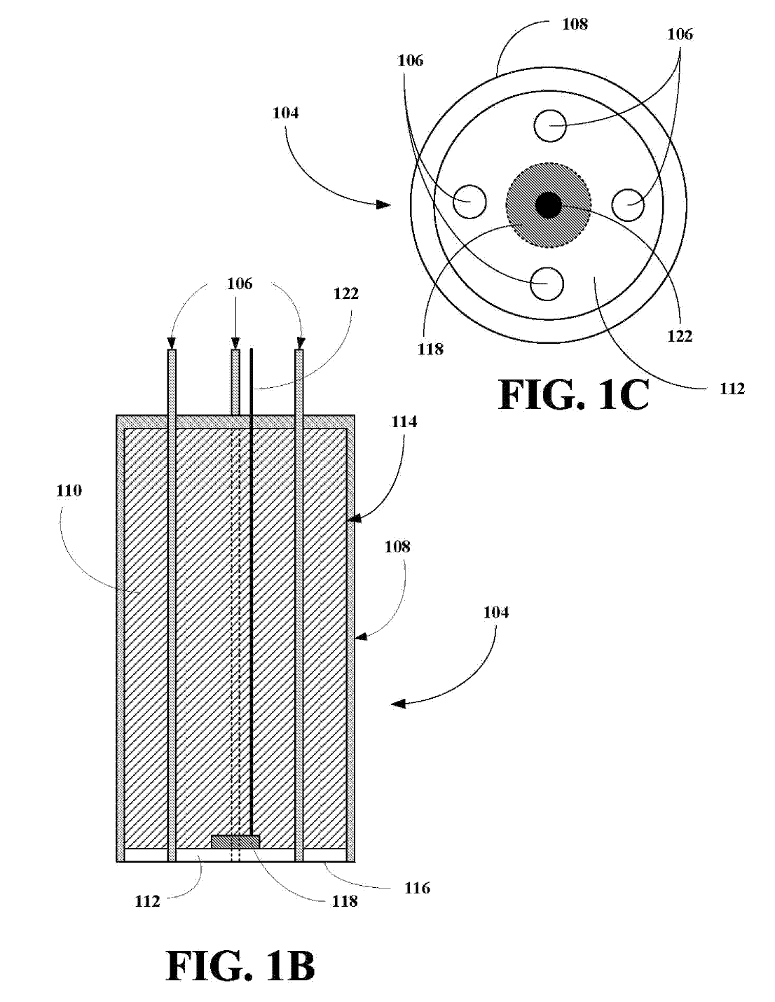

The experimental setup included an optoacoustic system generally 100 and shown in FIG. 1A. The system 100 includes a light source 102 such as a compact optical parametric oscillator (OPO) system (Opolette 532 II, Opotek Inc., Carlsbad, Calif.), which generates pulsed tunable infrared radiation having a wavelength range between about 680 and about 2440 nm, a pulse duration about 10 ns, and a repetition rate about 20 Hz. The light from the light source 102 was delivered to a probe 104 via four multi-mode optical fibers 106 having diameters of 1 mm. The probe 104 is an embodiment of an optoacoustic probe of this invention as shown in FIGS. 1B & C and arranged in a circular manner. The probe 104 included a housing 108. The housing 108 includes a backing 110 and an aluminum foil end 112. The four optical fibers 106 go through a probe interior 114 and terminates in a distal surface 116 of the probe 104. The probe 104 include a broad-band sensitive unfocused transducer 118 for detection of acoustic waves generated in the radial artery due to the optical pulses generated by the light source 102. The transducer 118 has a bandwidth of 3 MHz with a resonance frequency of 2 MHz and a sensitivity of 40 μV/Pa. The transducer 118 is made of piezo-ceramics having a very low planar coupling coefficient (<0.01) that minimizes acoustic ringing in the probe. In contrast to this new material, standard piezo-ceramic materials have high planar coupling coefficient and, hence, induce acoustic ringing which substantially complicates detection of the wide-band optoacoustic waves. Signals detected by the transducer 118 are fed to a low-noise amplifier 120 via a signal conduit 122 and then to a fast 100-MHz digitizer 124 (National Instruments Corp., Austin, Tex.) via a connection 126. An output signal is then forwarded to a laptop computer 128 via a connection 130. The computer 128 is used to control the OPO light source 102 via a connection 132 and for data acquisition and processing. Of course, the probe 104 can include more or less optical fibers for forwarding the light pulses from the light source 102 to a site of a patient's skin or body. Additionally, the probe 104 can include additional transducers to improve detection of the pressures pulses produced in the patient's skin or tissue site induced by the light pulses from the light source 102.

In this experiment we irradiated the skin of healthy volunteers over the radial artery and performed measurements at 16 different wavelengths from 680 to 1064 nm. The forearm was positioned in a custom-made hand holder to minimize movement. The probe placed was in contact with skin, and a thin layer of ultrasound gel was applied to ensure acoustic matching. We averaged 400 signals for every record to increase signal-to-noise-ratio (SNR). The laser fluence at the site of probing was about 4 mJ/cm² that was well below the maximum permissible exposure for skin in this spectral range.

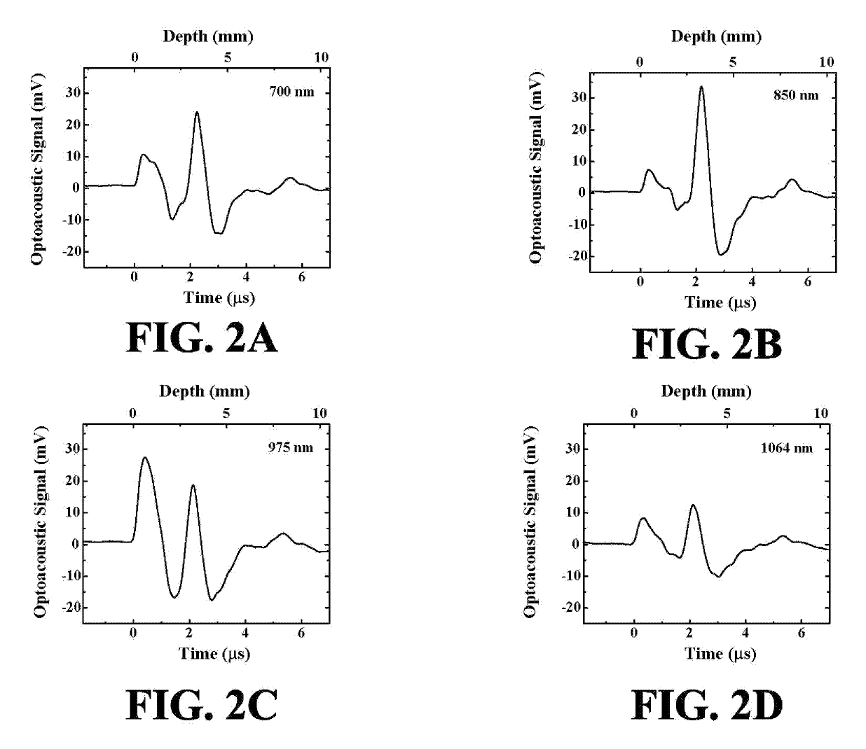

Referring now to FIG. 2, representative optoacoustic signals obtained from the wrist area of a healthy volunteer irradiated by OPO operating at different wavelengths are shown. The first peak reflects the absorption of light in skin, and the second peak is due to absorption in blood of the radial artery. At a wavelength λ=700 nm, the signal induced in the radial artery is slightly greater than the signal from skin; at λ=850 nm, the arterial signal greatly exceeds the skin signal; at λ=975 nm the amplitude of the arterial signal is considerably lower than that of the skin signal and at λ=1064 nm, both signals have low amplitudes.

These variations in the optoacoustic signal can be explained by considering tissue and blood dominant chromophores and spectral variations of their optical properties. In skin, the major absorbers in the specified spectral range are melanin and water, whereas in arterial blood it is oxyhemoglobin. The absorption of light in soft tissue between the skin surface and radial artery is primarily due to water content (the absorption by lipids at the considered wavelengths is much lower than that by water).

At λ=700 nm the absorption of oxyhemoglobin is relatively low, water absorption is negligible, and melanin absorption is quite high. That's why at this wavelength the amplitudes of both peaks are comparable with a slight prevalence of the peak from the radial artery. At λ=850 nm, the absorption of oxyhemoglobin is the highest, there is insignificant water absorption, and melanin absorbs much less than at shorter wavelengths. This explains the dominance of the arterial peak. At λ=975 nm, the high amplitude of the signal from overlying tissues (the first peak) is a manifestation of the water absorption band around λ=980 nm. At λ=1064 nm, the absorption of both oxyhemoglobin and water are low, and there is negligible melanin absorption, so both peaks in the signal are low.

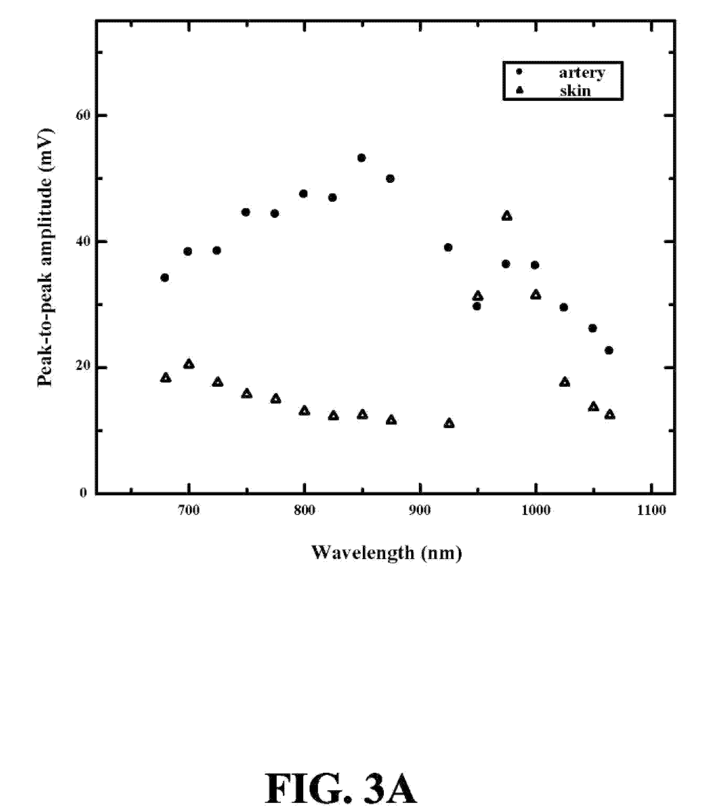

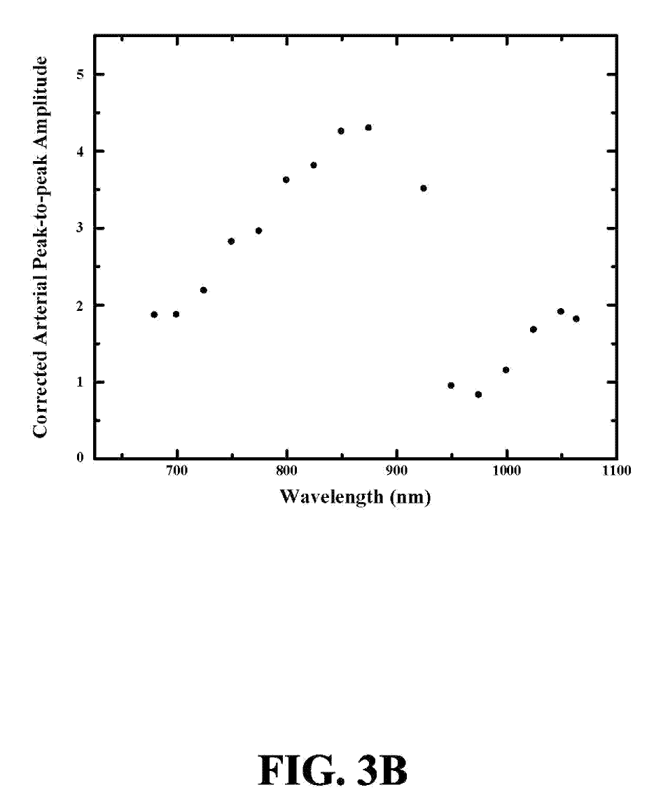

We calculated peak-to-peak amplitudes of the skin and arterial signals for all considered wavelengths. The results are shown in FIG. 3A. One can see that the amplitude of the skin signal (open triangles) is the lowest at wavelengths from 800 to 925 nm. In the same range, the arterial signal has quite high amplitude (solid circles). This means the light with the wavelengths within this range is least attenuated by overlying tissues and thus can be considered optimal for optoacoustic probing of superficial blood vessels. The spectral dependence of the skin signal amplitude has a distinctive peak at λ=975 nm due to water absorption. We corrected the arterial peak-to-peak amplitude by taking into account spectral response of our system. The results are shown in FIG. 3B. By analyzing the well-known absorption spectra of hemoglobin derivatives and water and the data obtained in this study, one can conclude that, in arterial blood, [HbOxy] is 90%, [Hb] is 10%, and [HbMet] is negligible (below 1%), while the water content in the overlying tissue is approximately 75%.

After the optimal spectral range was determined, we replaced the OPO in our experimental system with a nanosecond pulsed laser diode operating at λ=905 nm leaving all other elements of the system unchanged. The output power of this laser diode was 210 W with pulse duration of 100 ns.

First, we tested our laser-diode system in a phantom study. Our radial artery phantom was a clear plastic tube with an inner diameter of 2.4 millimeter (close to the diameter of a human radial artery). We filled the tube with fresh heparinized arterial sheep blood, which was centrifuged to increase [THb] up to 16 g/dL. To simulate soft tissue around the artery, we immersed the tube into 0.6% Intralipid solution (Baxter Healthcare Corp., Deerfield, Ill.) in distilled water. The optoacoustic probe was positioned 3 mm above the tube and was slightly immersed in the turbid solution to ensure acoustic contact. Then, we gradually diluted blood with saline and performed optoacoustic measurements after every dilution. Simultaneously, we directly measured blood parameters including [THb] with a standard CO-Oximeter (IL 682, Instrumentation Laboratories, Lexington, Mass.). To minimize the influence of melanin absorption and tissue scattering on signals from the radial artery, we chose a laser diode with the wavelength of 905 nm.

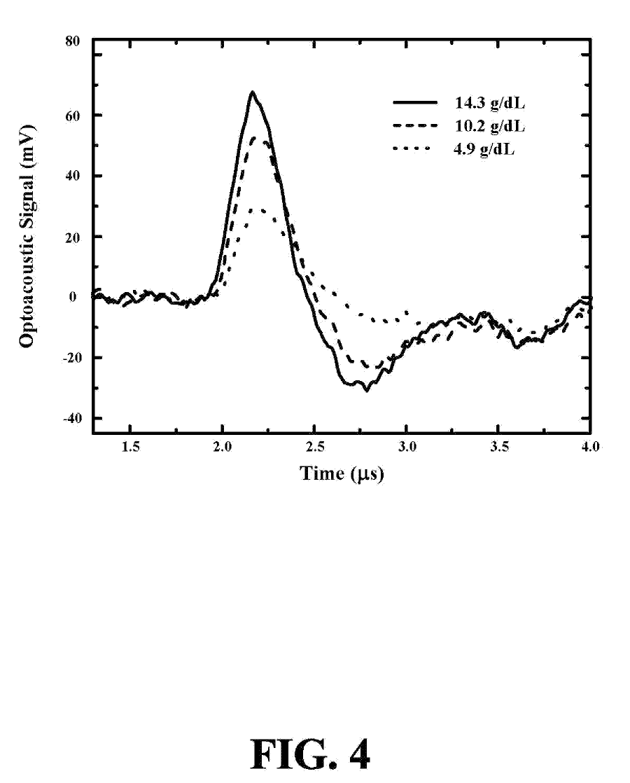

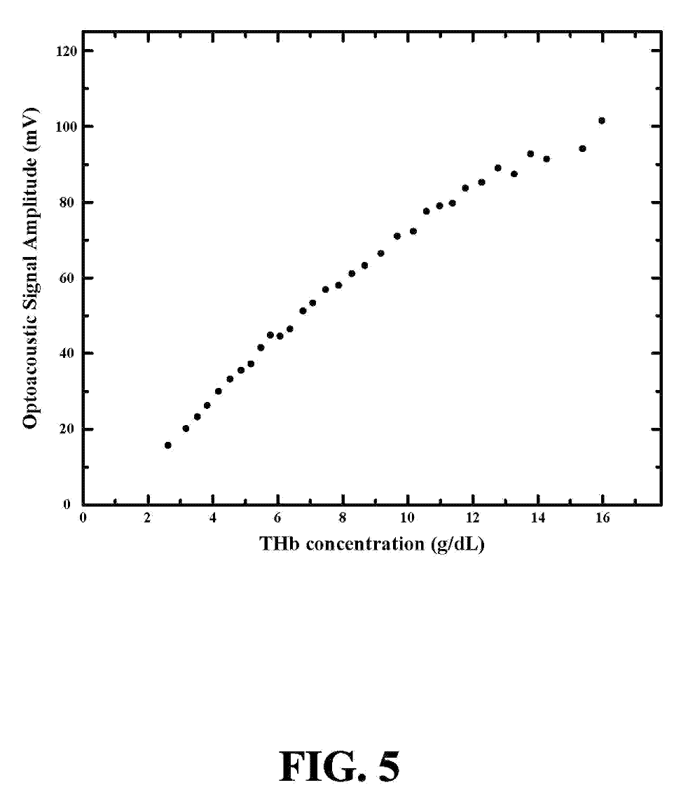

Referring now to FIG. 4, optoacoustic signals from sheep blood with varying [Thb] are shown: solid line 14.3 g/dL, dashed line 10.2 g/dL, and dotted line 4.9 g/dL. The signals were averaged 400 times to ensure suppression of electronic noise. One can see that the magnitude of the signal increases with [THb]. We calculated the peak-to-peak amplitude of this bipolar signal and plotted it vs. [THb] as shown in FIG. 5. The correlation is close to linear, at least for [THb] up to 13 g/dL. This fact allows for calibration of our optoacoustic system for absolute [THb] measurements, if a blood sample is taken at the beginning of a monitoring cycle (that is done for many patients anyway).

The next step was to test our laser-diode-based system in vivo. We secured a volunteer's forearm with Velcro straps in a custom-designed hand holder to minimize movement. The probe was positioned over the radial artery in contact with skin. We finely adjusted its alignment with the artery with a 3D translation stage.

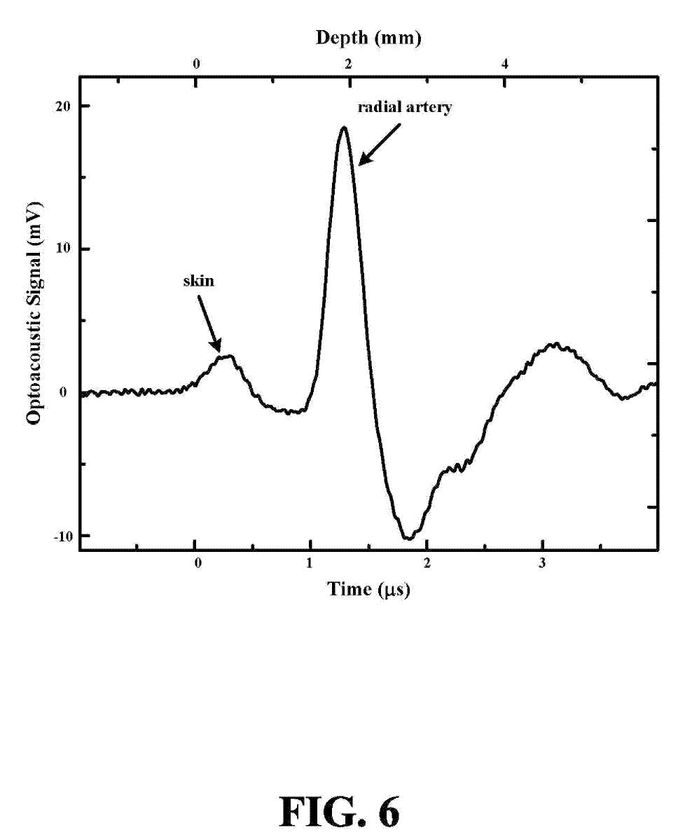

Referring now to FIG. 6, the signal from the radial artery of a healthy Caucasian volunteer obtained with the laser-diode-based optoacoustic system is shown. Again, the first peak represents the signal induced in skin, and the second peak is the signal generated in the radial artery. The peaks are well resolved, and the arterial peak greatly exceeds the skin peak in amplitude, just as we expected to have, when choosing the operating wavelength of 905 nm. We obtained similar signals from the radial arteries of two other volunteers. What is most remarkable, despite the relatively low energy of laser diode, the signal-to-noise ratio in the signals was very high. After averaging of 400 signals (that took about 30 seconds) the SNR varied from 260 to 390 depending on the peak-to-peak amplitude of the arterial signal (we calculated the SNR as the peak-to-peak amplitude of the arterial signal divided by the root-mean-square of the noise magnitude). This fact confirms the potential of our laser-diode-based optoacoustic system to be used for clinical monitoring of blood parameters. Indeed, the signal shown in FIG. 6 had an SNR of 390 and was obtained from a volunteer with [THb]=16 g/dL. It means, even for [THb] as low as 5 g/dL the SNR will still have a high value of about 120.

The oxygenation of arterial blood in healthy individuals and most patients is high, 95-98%. The variation of oxygenation within this range does not cause significant change in the absorption coefficient of hemoglobin. For instance, for wavelengths within the optimal spectral range for optoacoustic [THb] monitoring (800-925 nm) the difference between absorption coefficients of hemoglobin with oxygenation level of 95% and 98% does not exceed 1% and can be neglected.

The monitoring of blood parameters can be performed not only in arteries, but in veins, too. The concentration of oxyhemoglobin is lower in veins than in arteries and varies greatly depending on many factors. To avoid the influence of variable oxyhemoglobin saturation of blood on optoacoustic signals, the measurements can be performed at a wavelength of 805 nm, which is an isobestic point of blood absorption spectrum (the absorption coefficient of blood does not depend on the blood oxygenation at this wavelength).

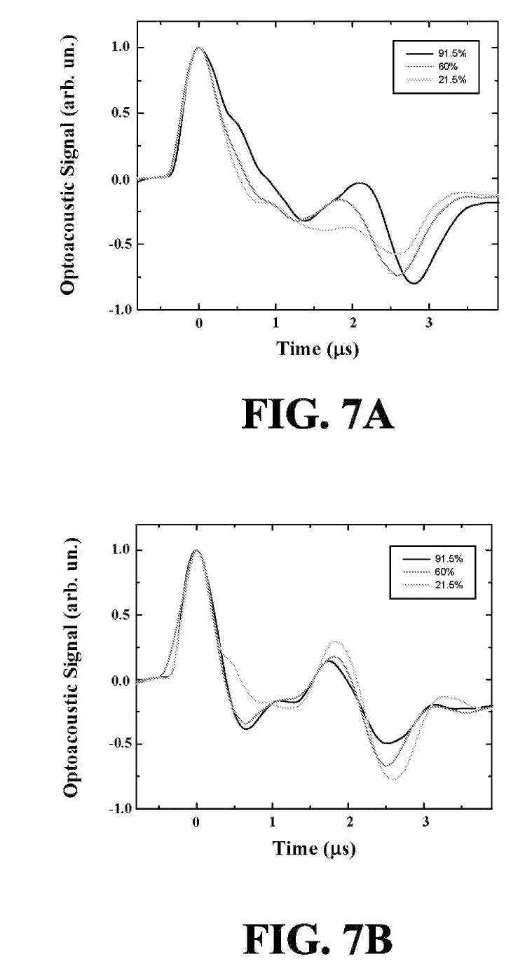

The peak-to-peak amplitude measurement can be used for accurate monitoring not only [THb], but all the other blood parameters. For instance, it can be used for accurate measurement of blood oxygenation. The following data show accurate monitoring of cerebral venous blood oxygenation by measuring the peak-to-peak amplitude of the optoacoustic signal induced superior sagittal sinus (SSS), a large central cerebral vein. FIGS. 7A & B shows typical optoacoustic signals recorded at 1064 nm and 700 nm from sheep SSS at different oxygenation (91.5% (black), 21.5% (light grey), and 60% (dark grey)). The first peak (at t=0 μs) is induced in skull, while the second peak (at t=2 μs) was induced is the SSS due to absorption of light by blood circulating in the SSS. These data demonstrate the high resolution of the optoacoustic technique that allows for direct probing of blood vessels through optically and acoustically scattering tissues. The signal from the SSS at 700 nm as shown in FIG. 7B decrease with oxygenation that is consistent with the known spectrum for oxy- and deoxygenated blood. In contrast, at 1064 nm as shown in FIG. 7A the signal from the SSS increases dramatically with oxygenation because absorption coefficient of oxygenated hemoglobin is substantially higher than that of deoxygenated.

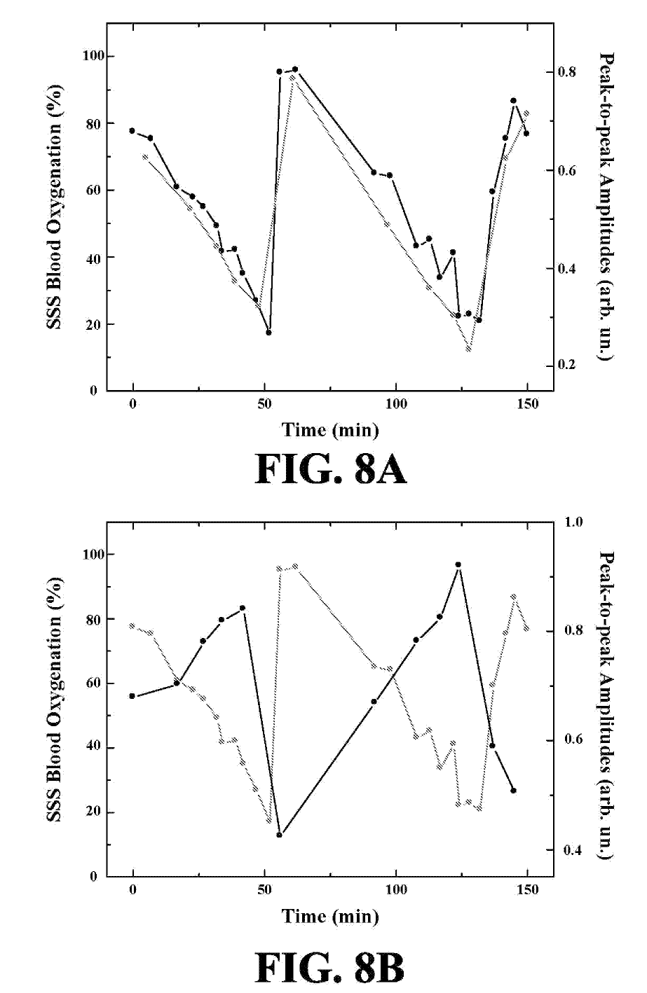

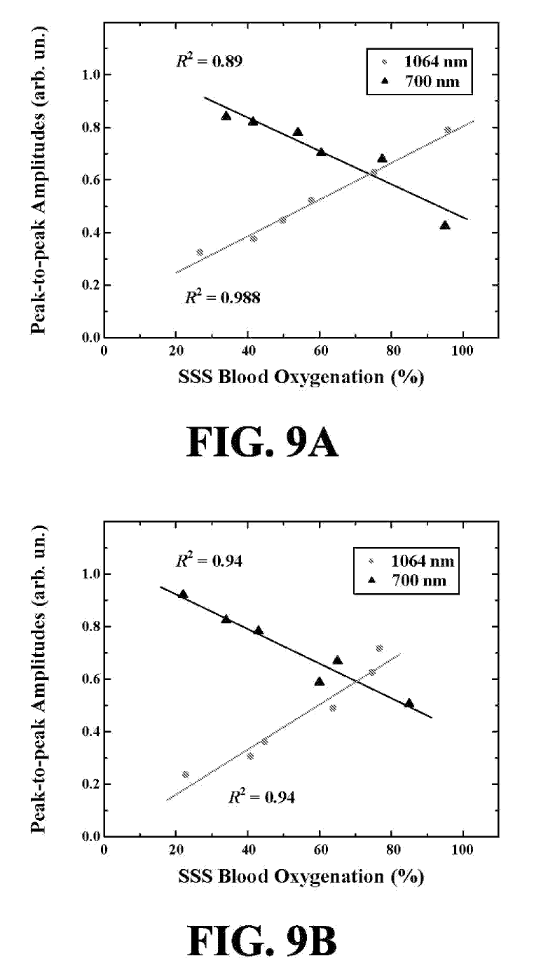

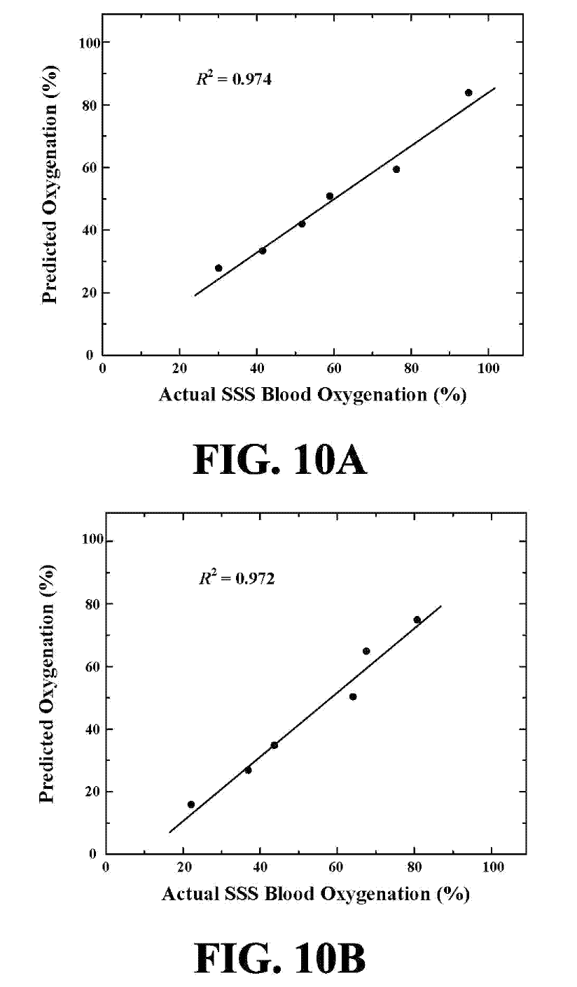

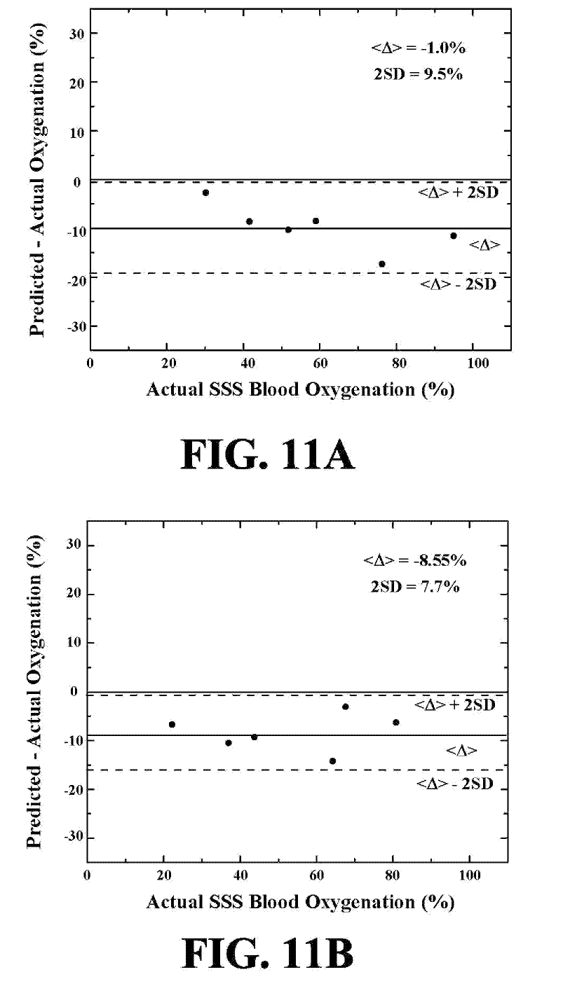

The optoacoustic signal peak-to-peak amplitudes measured from the sheep SSS during variation of blood oxygenation in two cycles are presented for 1064 nm and 700 nm as shown in FIG. 8A & B, respectively. The amplitudes were normalized by the peak-to-peak amplitudes recorded at 805 nm. The peak-to-peak amplitudes closely followed actual SSS blood oxygenation measured with CO-Oximeter for both wavelengths as shown in FIGS. 9A & B, respectively). Moreover, the amplitudes were linearly dependent on blood oxygenation with high correlation: R²=0.890-0.988 for the both first as shown in FIG. 9A and the second cycle as shown in FIG. 9B. We predicted the SSS blood oxygenation by using our in vitro calibration curve and the ratio of the peak-to-peak amplitude measured from the sheep SSS in vivo. FIG. 10 shows the optoacoustically predicted SSS blood oxygenation vs. actual blood oxygenation measured with the CO-Oximeter for the first (a) and second (b) cycles. The correlation between the optoacoustically predicted SSS blood oxygenation and actual blood oxygenation was very high for both the cycles (R²=0.974 and 0.972). To estimate the bias and standard deviation of the optoacoustic measurements, we calculated the difference between the optoacoustically predicted and actual oxygenation measured with CO-Oximeter. FIGS. 11A & B show the bias and standard deviation for the first and the second cycles, respectively. These data demonstrate that the accuracy of the optoacoustically measured blood oxygenation is approaching that of invasive measurements.

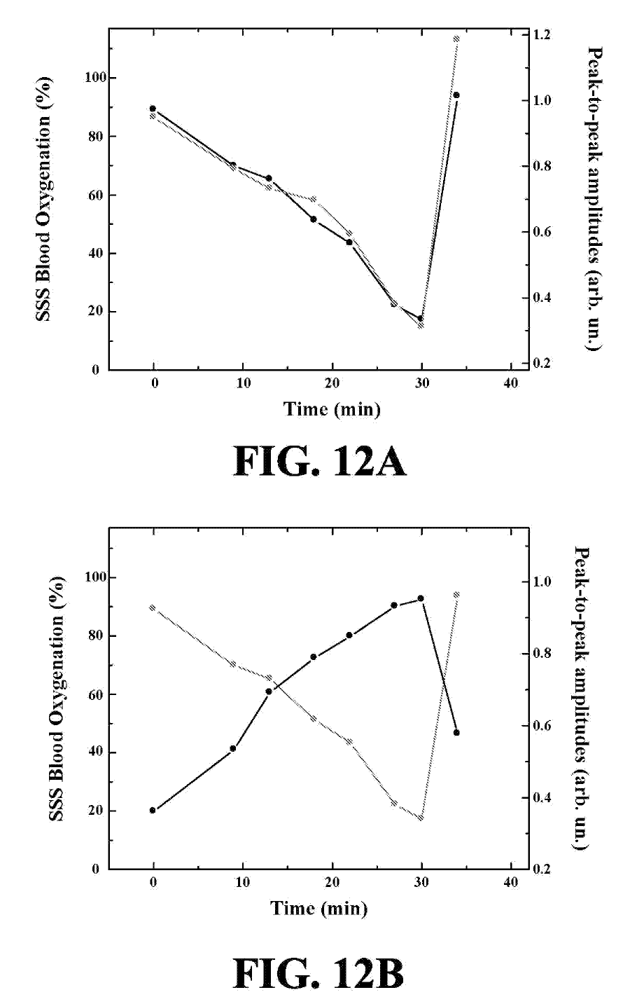

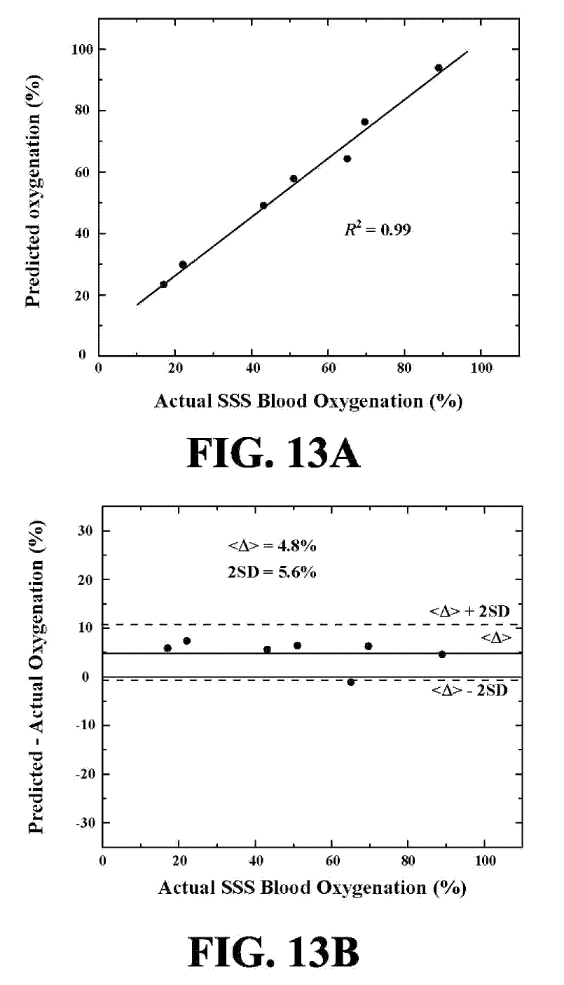

The major confounding variables in optoacoustic measurements in sheep were due to motion artifacts. FIGS. 12A & B show SSS blood oxygenation and optoacoustic signal amplitude at 1064 nm and 700 nm, respectively, that were measured when the sheep had minimal motion. The signal amplitude correlated better as shown in FIG. 13A than that measured at typical motion of the sheep. This resulted in lower bias and standard deviation (4.8% and 5.6%, respectively) as shown in FIG. 13B compared to that for the sheep with typical motion as shown in FIG. 11A & B.

To minimize influence of motion artifacts, one can use scanning of the optoacoustic probe on the skin surface over the blood vessel.

### First Derivative of Normalized Signal

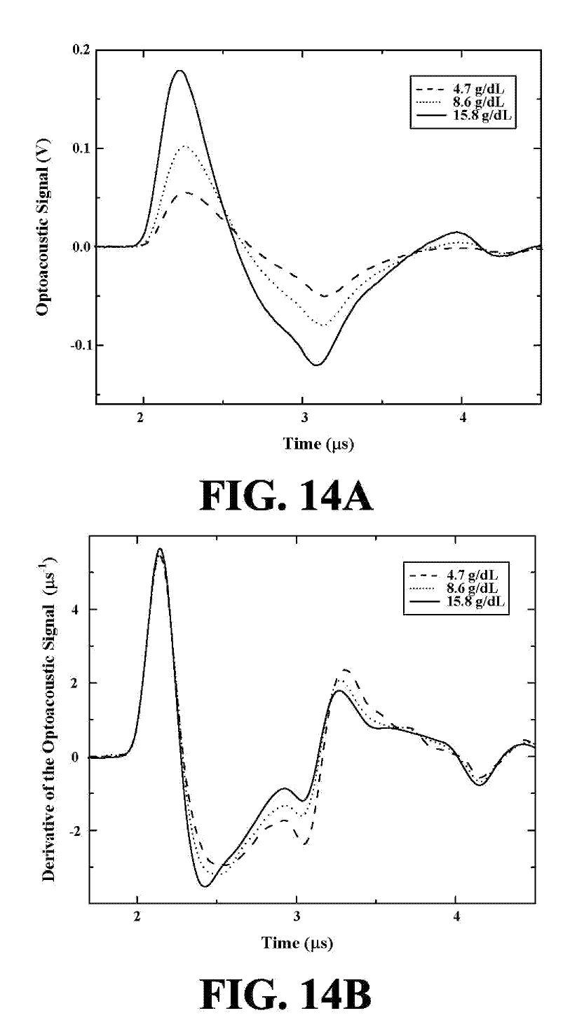

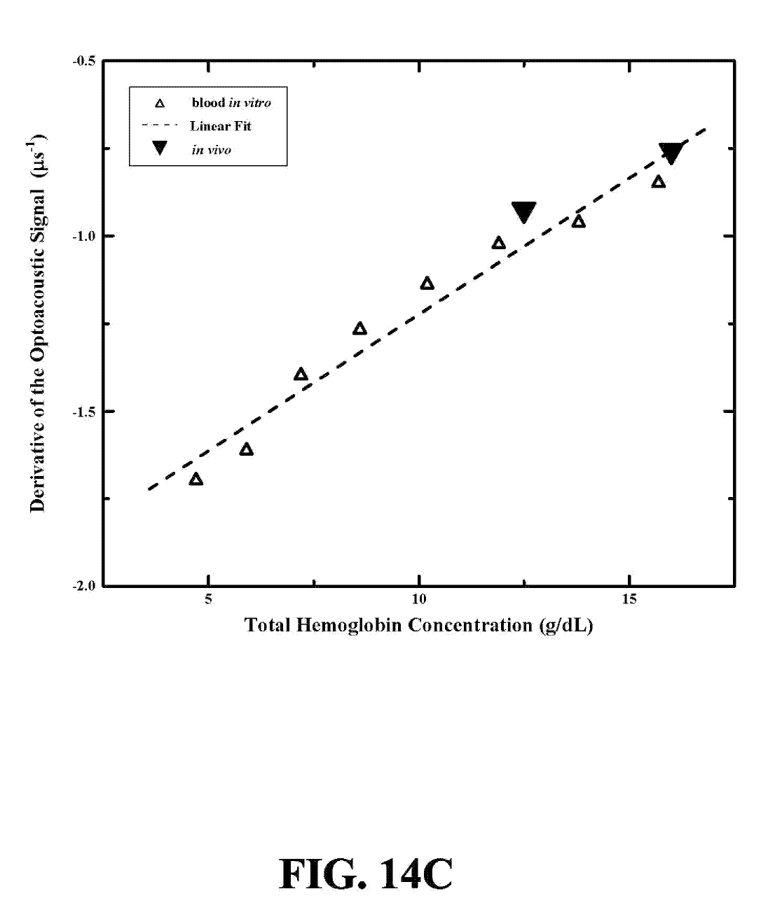

To monitor the physiological variables using the first derivative of normalized signal, in particular, from smaller blood vessels, one needs to use optoacoustic probes with higher frequency. We built a wide-band optoacoustic probe with 10-MHz frequency range and used it in in vitro and in vivo studies on monitoring of the blood variables using the measurement of the derivative of the normalized signal. FIG. 14A shows typical optoacoustic signals recorded from the radial artery phantom which resembles geometry of the radial artery and optical properties of overlying tissues. Arterial blood at different [THb] was used in the study. We normalized the signal amplitude to 1.0 and then calculated the first derivative of the normalized signals as shown in FIG. 14B. The derivatives were dependent on [THb], in particular, at the maximum located between 2.7 and 3.0

### Scanning and Array to Reduce Influence of Motion Artifacts

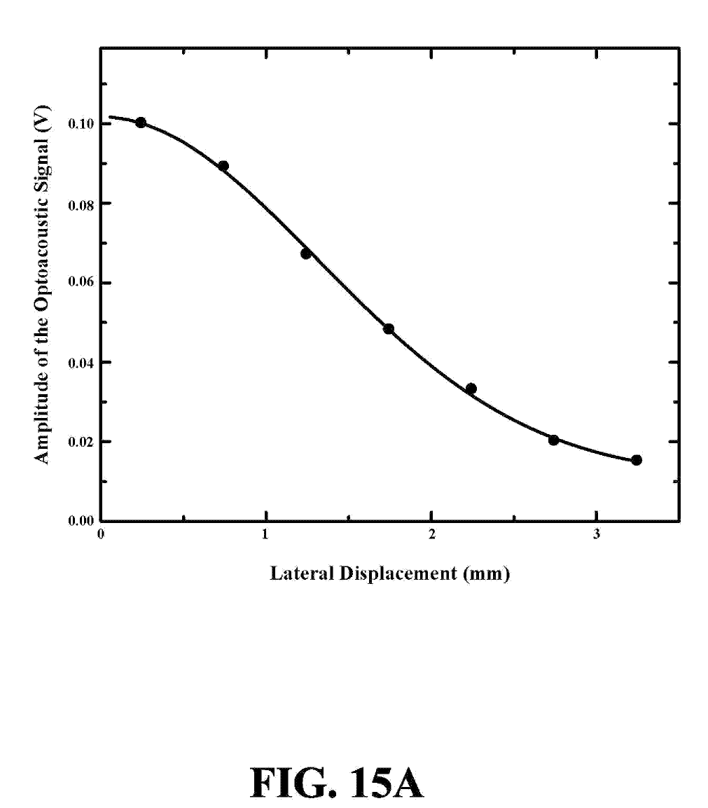

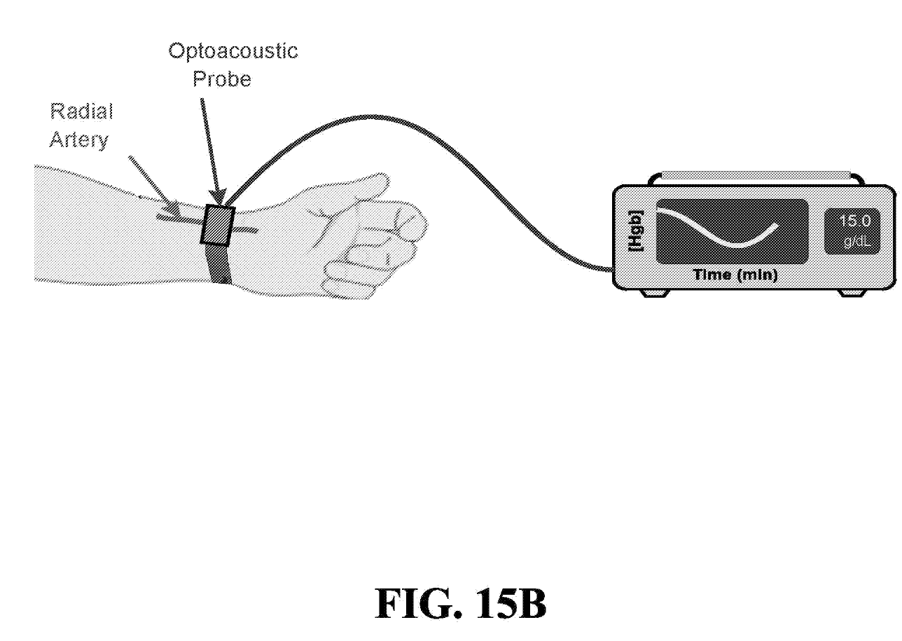

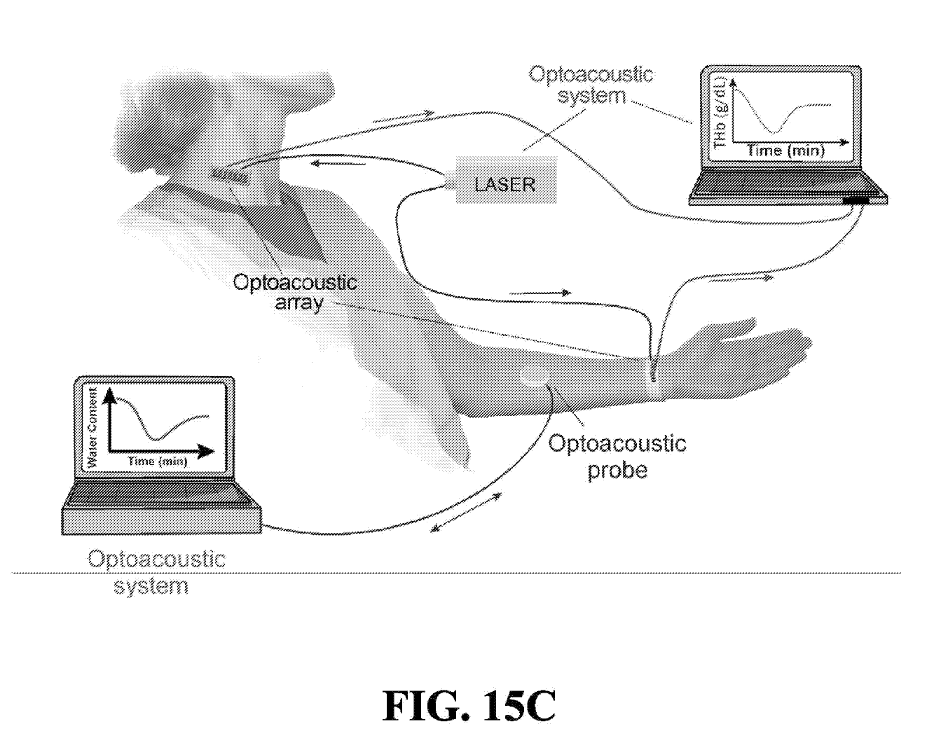

Motion of tissues in the probed areas results in displacement of the optoacoustic probe with respect to the blood vessel and hence reduces the accuracy of optoacoustic monitoring. To minimize the influence of motion artifacts, we propose to use scanning of the optoacoustic probe on the skin surface over the blood vessel. In particular, the scanning improves accuracy of optoacoustic monitoring based on amplitude and peak-to-peak amplitude measurements because both these amplitudes are dependent on alignment of the probe with respect to the blood vessel. FIG. 15A shows dependence of the amplitude of the optoacoustic signal recorded from the radial artery phantom at different lateral displacement (misalignment) of the probe. The amplitude decreases substantially with lateral displacement. Therefore, using scanning one can adjust the probe position to the maximum of the blood vessel signal and the influence of the motion artifacts will be reduced.

FIG. 15B shows an example of an optoacoustic system with a probe scanning over the radial artery that can be used for monitoring of the physiologic variables. The probe scanning can be performed in lateral direction and alone the artery. Angular scanning can be used too to find best alignment of the probe with respect to the blood vessel.

Instead of a scanning system an optoacoustic array can be used. FIG. 15C shows an example of an array-based optoacoustic system for monitoring of physiologic variables in the arterial blood (in the radial artery) and in the venous blood (in the internal jugular vein). Each array has multiple acoustic detectors and either multiple fibers (beams) or a single fiber (beam). The array allows for detection of the best signal from the blood vessel without scanning because the detector above the blood vessel will provide best signal that can be used for monitoring of the variables. For monitoring of other physiologic variables such as content of water, myoglobin, lactate, fat, protein, calcium, etc., one can use also an optoacoustic system without array or scanning and only with one acoustic detector as shown in FIG. 15C.

### ICG Data

ICG has a maximum of absorption around 800-805 nm. We designed and built a laser diode-based optoacoustic system to measure ICG concentration for monitoring of the CO, CI, BV and hepatic function. The laser diode operates at 805 nm. We performed optoacoustic measurement using the system in whole arterial blood in vitro at clinically relevant concentrations of ICG. The measurements were performed in a 10-mm cuvette in the transmission mode, i.e., the laser irradiation and optoacoustic wave detection were performed from the opposite sides.

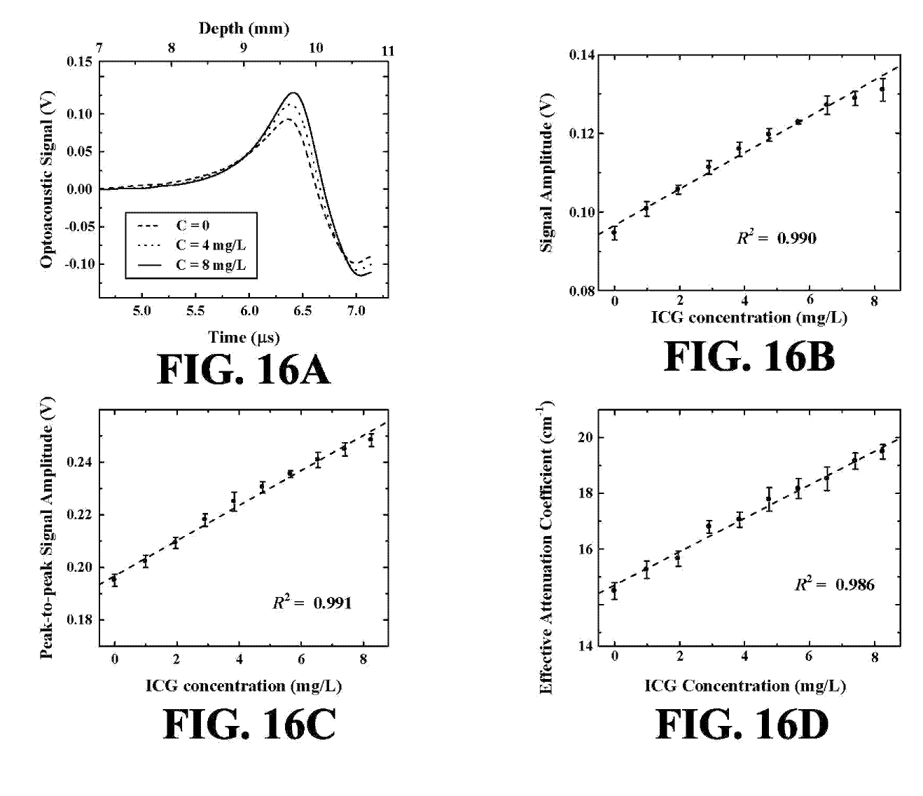

FIG. 16A shows typical optoacoustic signals recorded from the blood at different ICG concentration. Both amplitude and the temporal profile of the signal are dependent on the ICG concentration. We measured the signal amplitude, peak-to-peak amplitude, and the effective attenuation coefficient which was derived from the exponential profiles of the signals. The amplitude as shown in FIG. 16B and the peak-to-peak amplitude as shown in FIG. 16C increase with ICG concentration with high correlation coefficients (R²=0.990 and 0.991, respectively). The effective attenuation coefficient of blood presented in FIG. 16D also increases linearly with ICG concentration and the correlation coefficient is high: R²=0.986. It should be noted that absorption by hemoglobin produces the optoacoustic signal at ICG concentration=0 mg/dL. That's why the signal amplitude, peak-to-peak amplitude, and the effective attenuation coefficient are not equal to zero without the ICG. These data demonstrate that the optoacoustic technique is capable of sensitive detection of changes in the ICG concentration in blood and therefore, can be used for accurate monitoring of CO, CI, BV and hepatic function.

### Blood Pressure Measurements

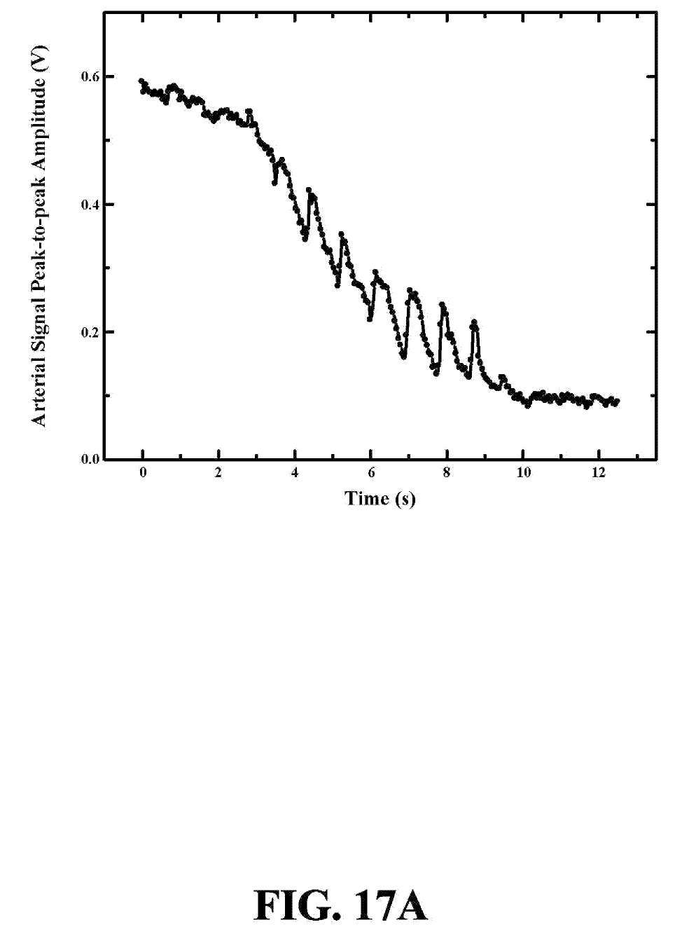

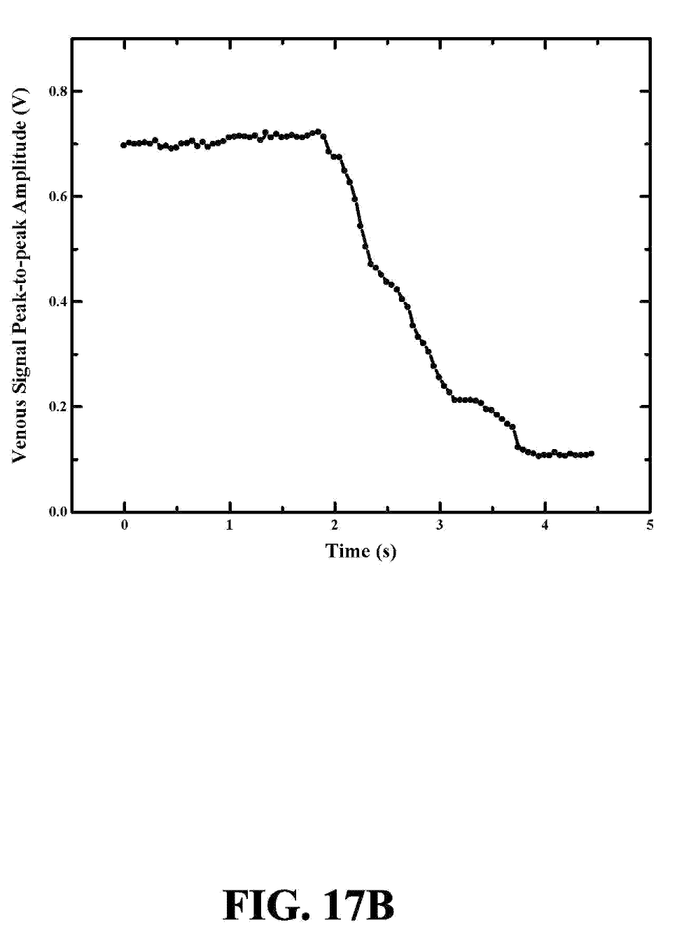

FIG. 17A depicts peak-to-peak amplitude measured in real time from the radial artery during variation of external pressure exerted by the optoacoustic probe. First, at low pressure (up to the 3^{rd} second), the optoacoustic signal peak-to-peak amplitude was high and pulsation of the peak-to-peak amplitude was negligible. When the pressure exerted on the artery was between diastolic and systolic pressure (between the 3^{rd} and 10^{th} seconds), the peak-to-peak amplitude was pulsating. When the pressure exerted on the artery exceeded the systolic pressure (after 10^{th} second), the pulsations disappeared because there was no blood in the artery. The residual peak-to-peak amplitude was detected from other tissues (not from blood) at this depth. Therefore, one can measure the arterial pressure by detecting the pulsations of the peak-to-peak amplitude. The pressure at which the optoacoustic signal pulsations start is diastolic, while the pressure at which the pulsations cease is systolic.

FIG. 17B depicts peak-to-peak amplitude measured in real time from a wrist vein during variation of external pressure exerted by the optoacoustic probe. First, at low pressure (up to t=1.9 s), the optoacoustic signal peak-to-peak amplitude was high. When the pressure exerted on the vein was increased (between 1.9 s and 3.7 s), the peak-to-peak amplitude decreased. When the pressure exerted on the artery exceeded the venous pressure (after t=3.7 s), the signal from the vein disappeared because there was no blood in the vein. The residual peak-to-peak amplitude was detected from other tissues (not from blood) at this depth. Therefore, one can measure the venous pressure by detecting the peak-to-peak amplitude. The pressure at which the optoacoustic signal from the vein disappears is venous pressure.

### REFERENCES CITED IN THIS INVENTION

The following references were cited herein:
1. Ganz W, Donoso R, Marcus H S, Forrester J S, Swan H J. “A new technique for measurement of cardiac output by thermodilution in man.” Am J Cardiol. 1971 Apr.; 27(4):392-6.
2. Montgomery W H, Hanson III C W. “Pulmonary Artery Catheter Education Program: A Progress Report on This New Collaborative Educational Tool.” ASA Newsletter, August 2002; 66.
3. Lapinsky S E, Richards G A. “Pro/con clinical debate: pulmonary artery catheters increase the morbidity and mortality of intensive care unit patients. Crit Care. 2003 Apr.;7(2):101-3.
4. Kern J W, Shoemaker W C. Meta-analysis of hemodynamic optimization in high-risk patients. Crit Care Med. 2002 Aug.;30(8): 1686-92.
5. Lobo S M, Salgado P F, Castillo V G, Borim A A, Polachini C A, Palchetti J C, Brienzi S L, de Oliveira G G. Effects of maximizing oxygen delivery on morbidity and mortality in high-risk surgical patients. Crit Care Med. 2000 Oct.;28(10):3396-404.
6. Davis J W, Davis I A, Bennink L D, Bilello J F, Kaups K L, Parks S N. Are Automated Blood Pressure Measurements Accurate in Trauma Patients? J Trauma 2003;55:860.
7. J. T. Kuenstner, K. H. Norris, “Spectrophotometry of human hemoglobin in the near infrared region from 1000 to 2500 nm,” Journal of Near Infrared Spectroscopy, 1994, vol. 2, pp. 59-65.
8. J. Lee, N. El-Abaddi, A. Duke, A. E. Cerussi, M. Brenner, B. J. Tromberg, “Noninvasive in vivo monitoring of methemoglobin formation and reduction with broadband diffuse optical spectroscopy,” Journal of Applied Physiology, 2006, vol. 100, pp. 615-622.
9. N. Yaroslavsky, I. V. Yaroslavsky, T. Goldbach, H.-J. Schwarzmaier, “The optical properties of blood in the near infrared spectral range,” Proceedings of SPIE, vol. 2678, pp. 314-324 (1996).
10. L. L. Randeberg, J. H. Bonesronning, M. Dalaker, J. S. Nelson, L. O. Svaasand, “Methemoglobin formation during laser induced photothermolysis of vascular skin lesions,” Lasers in Surgery and Medicine, 2004, vol. 34, pp. 414-419.
11. B. L. Horecker, “The absorption spectra of hemoglobin and its derivatives in the visible and near infra-red regions,” Journal of Biological Chemistry, 1943, vol. 148, pp. 173-183.
12. Zwart, A. Buursma, E. J. van Kampen, W. G. Zijlstra, “Multicomponent analysis of hemoglobin derivatives with a reversed-optics spectrophotometer,” Clinical Chemistry, 1984, vol. 30, no. 3, pp. 373-379.
13. W. G. Zijlstra, A. Buursma, W. P. Meeuwsen-van der Roest, “Absorption spectra of human fetal and adult oxyhemoglobin, de-oxyhemoglobin, carboxyhemoglobin, and methemoglobin,” Clinical Chemistry, 1991, vol. 37, no. 9, pp. 1633-1638.
14. Esenaliev R. O., Larina I. V., Larin K. V., Deyo D. J., Motamedi M., Prough D. S. Optoacoustic Technique for Noninvasive Monitoring of Blood Oxygenation: A Feasibility Study. Applied Optics, 2002, 41:4722-31.
15. Petrova I. Y., R. 0. Esenaliev, Y. Y. Petrov, H.-P. F. Brecht, C. H. Svensen, J. Olsson, D. J. Deyo, D. S. Prough, “Optoacoustic monitoring of blood hemoglobin concentration: a pilot clinical study”, Optics Letters, vol. 30, no. 13, pp. 1677-1679, July 2005.
16. Esenaliev R. O., Y. Y. Petrov, O. Hartupf, D. J. Deyo, D. S. Prough, “Continuous, noninvasive monitoring of total hemoglobin concentration by an optoacoustic technique,” Applied Optics (Optical Technology and Biomedical Optics), vol. 43, no. 17, pp. 3401-3407, 2004.
17. Petrov Y. Y., D. S. Prough, D. J. Deyo, M. Klasing, M. Motamedi, R. 0. Esenaliev, “Optoacoustic, noninvasive, real-time, continuous monitoring of cerebral blood oxygenation: an in vivo study in sheep,” Anesthesiology, vol. 102, no. 1, pp. 69-75, 2005.
18. Petrov Y. Y., Petrova I. Y., Patrikeev I. A., Esenaliev R. O., Prough D. S. Multiwavelength optoacoustic system for noninvasive monitoring of cerebral venous oxygenation: a pilot clinical test in the internal jugular vein. Optics Letters, v. 31 (12) pp. 1827-1829, 2006.

A piezo-ceramic with low planar coupling coefficient. It is a modified lead titanate piezo-ceramic Nova 3A or Nova 7A manufactured by Keramos, Inc., Indianapolis, Ind. I think it might be a good idea to be specific and present an example.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. An optoacoustic system for absolute or relative, accurate, and real-time or near real-time measuring or monitoring of a variety of at least one diagnostic variable comprising:
a pulsed light source,
a light source delivery subsystem comprising one optical fiber or a plurality of optical fibers or a light delivery subsystem comprising conventional (non-fiber) optical elements,
a probe including a transducer for detection of acoustic waves generated in a site or tissue of a patient due to the optical pulses generated by the light source,
a registration subsystem to acquire, analyze, and process data from the transducer and to display or send data containing at least one absolute or relative value of the diagnostic variable or changes in the value of the diagnostic variable.

2. The system of claim 1, wherein the registration system comprises:
a low-noise amplifier is connected to the transducer via a signal conduit,
a digitizer is connected to the amplifier via a connection,
a laptop computer is connected to the digitizer via a connection and to the light source via a connection, where the computer controls the light source and acquires, analyzes and processes data from the transducer via and amplifier and digitizer.

3. The system of claim 1, wherein the light source is capable of generating at least one pulse with duration in the range between about 1 femtosecond and about 10 microseconds and with at least one wavelength in the range between about 200 nm and about 20,000 nm; and preferably with duration in the range between about 0.1 nanoseconds and about 200 nanoseconds and with wavelength in the range between about 600 nm and 2,000 nm.

4. The system of claim 1, wherein the fiber comprises or fibers comprise multi-mode optical fibers.

5. The system of claim 1, wherein the fiber has or fibers have diameters in the range between about 10 microns to about 2 mm.

6. The system of claim 1, wherein the fiber has or fibers have diameters in the range between about 100 microns and about 1 mm.

7. The system of claim 1, wherein an array of the transducers is used for more accurate monitoring.

8. The system of claim 1, wherein the transducer is a broad-band sensitive focused or unfocused transducer containing at least one conventional piezo-element or at least one peizo-element with low planar coupling coefficient or containing a subsystem for optical detection of acoustic waves.

9. The system of claim 1, wherein the transducer has a bandwidth in the range between about 0.01 MHz to about 100 MHz; and preferably in the range between about 0.05 MHz and about 10 MHz.

10. The system of claim 1, where in the probe is scanned over tissue surface in at least one direction or in at least one angular direction, or in mixtures or combinations thereof to provide more accurate monitoring.

11. The system of claim 1, where in the probe is inserted in a hollow organ to better access blood vessel or tissue of interest.

12. The system of claim 1, wherein the variable is selected from the group consisting of: (1) circulating blood volume, cardiac output; (2), cardiac index, systemic oxygen delivery; (3) concentrations of hemoglobin derivatives (e.g., carboxyhemoglobin, reduced hemoglobin, oxygenated hemoglobin, and methemoglobin), total hemoglobin concentration, concentrations of lactate, myoglobin, cholesterol, body pigments, and exogenous dyes; (4) content in tissues of water, fat, protein, calcium, and blood; (5) density of hard and soft tissues; (6) blood pressure in arteries, arterioles, veins, capillaries; and (7) mixtures or combinations thereof.

13. The system of claim 12, wherein the hemoglobin derivatives are selected from the group consisting of carboxyhemoglobin [HbCO], reduced hemoglobin [Hb], oxygenated hemoglobin [HbOxy], and methemoglobin [HbMet].

14. The system of claim 1, wherein the measuring comprises a single measurement, a periodic measurement, a semi-continuous, or a continuous measurement of one or more of the parameters.

15. A method for optoacoustic monitoring of important parameters comprising the step of:
placing an optoacoustic probe of an optoacoustic system against a site of an animal including an human, where the system includes:
a pulsed light source,
a light source delivery subsystem comprising one optical fiber or a plurality of optical fibers or a light delivery subsystem comprising conventional (non-fiber) optical elements,
a probe including a transducer for detection of acoustic waves generated in a site or tissue of a patient due to the optical pulses generated by the light source,
a registration subsystem to acquire, analyze, and process data from the transducer and to display or send data containing at least one absolute or relative value of the diagnostic variable or changes in the value of the diagnostic variable
irradiating the site with light pulses emanating from the light delivery subsystem;
detecting a pressure response signal from the site via the transducer,
receiving a pressure response signal from the site,
detecting the pressure response signal in a detector,
measuring at least one of the pressure response signal parameters selected from a group consisting of a peak-to-peak amplitude of the detected response signal, an amplitude of the detected response signal, a first derivative of normalized response signal, a slope of the response signal, or a mixtures or combinations thereof.

16. The method of claim 15, wherein the registration system comprises:
a low-noise amplifier is connected to the transducer via a signal conduit,
a digitizer is connected to the amplifier via a connection,
a laptop computer is connected to the digitizer via a connection and to the light source via a connection, where the computer controls the light source and acquires, analyzes and processes data from the transducer via and amplifier and digitizer.

17. The method of claim 15, wherein the light source is capable of generating at least one pulse with duration in the range between about 1 femtosecond and about 10 microseconds and with at least one wavelength in the range between about 200 nm and about 20,000 nm; and preferably with duration in the range between about 0.1 nanoseconds and about 200 nanoseconds and with wavelength in the range between about 600 nm and 2,000 nm.

18. The method of claim 15, wherein the fiber comprises or fibers comprise multi-mode optical fibers.

19. The method of claim 15, wherein the fiber has or fibers have diameters in the range between about 10 microns to about 2 mm.

20. The method of claim 15, wherein the fiber has or fibers have diameters in the range between about 100 microns and about 1 mm.

21. The method of claim 15, wherein an array of the transducers is used for more accurate monitoring.

22. The method of claim 15, wherein the transducer is a broad-band sensitive focused or unfocused transducer containing at least one conventional piezo-element or at least one peizo-element with low planar coupling coefficient or containing a subsystem for optical detection of acoustic waves.

23. The method of claim 15, wherein the transducer has a bandwidth in the range between about 0.01 MHz to about 100 MHz; and preferably in the range between about 0.05 MHz and about 10 MHz.

24. The method of claim 15, where in the probe is scanned over tissue surface in at least one direction or in at least one angular direction, or in mixtures or combinations thereof to provide more accurate monitoring.

25. The method of claim 15, where in the probe is inserted in a hollow organ to better access blood vessel or tissue of interest.

26. The method of claim 15, wherein the variable is selected from the group consisting of: (1) circulating blood volume, cardiac output; (2), cardiac index, systemic oxygen delivery; (3) concentrations of hemoglobin derivatives (e.g., carboxyhemoglobin, reduced hemoglobin, oxygenated hemoglobin, and methemoglobin), total hemoglobin concentration, concentrations of lactate, myoglobin, cholesterol, body pigments, and exogenous dyes; (4) content in tissues of water, fat, protein, calcium, and blood; (5) density of hard and soft tissues; (6) blood pressure in arteries, arterioles, veins, capillaries; and (7) mixtures or combinations thereof.

27. The method of claim 26, wherein the hemoglobin derivatives are selected from the group consisting of carboxyhemoglobin [HbCO], reduced hemoglobin [Hb], oxygenated hemoglobin [HbOxy], and methemoglobin [HbMet].

28. The method of claim 15, wherein the measuring comprises a single measurement, aperiodic measurement, a semi-continuous, or a continuous measurement of one or more of the parameters.
